(19) United States
(12) Patent Application Publication
(10) Pub. No.: US 2026/0263252 A1
(43) Pub. Date: Sep. 10, 2026

(54) DYNAMIC HIP SPICA SYSTEM, METHOD FOR PROVIDING DYNAMIC ORTHOSIS

(71) Applicants: Lauren M. Roethlisberger, Ashwaubenon, WI (US); Mariah M. Keopple, New Richmond, WI (US)

(72) Inventors: Lauren M. Roethlisberger, Ashwaubenon, WI (US); Mariah M. Keopple, New Richmond, WI (US)

(21) Appl. No.: 19/069,500

(22) Filed: Mar. 4, 2025

(51) Int. Cl. A61F 5/01; A41D 27/00

(52) U.S. Cl. CPC A61F 5/0104; A41D 27/00; A61F 5/0193

(57) ABSTRACT

The invention provides a garment for providing orthosis, the garment comprising: a foundation garment defining a crotch, a waistband, front leg regions and back leg regions; hook and pile fasteners overlying regions of the shorts; and reversibly deformable elongated substrate removably received by the fasteners. Also provided is a method for providing orthosis, the method comprising applying pressure to effected areas of a patient overlaid with indicia.

### BACKGROUND OF THE INVENTION

### 1. Field of the Invention

This invention relates to therapeutic clothing and physical therapy methods, and more specifically, this invention relates to an orthotic and a method for providing orthosis.

### 2. Background of the Invention

The aiding of disorders of the limbs and/or spine by use of braces and other devices is common. These tools normally are used in combination with appropriate rehab/treatment plans in order to reestablish or allow continuation of desired physical activity by a patient.

There are several types of garments and methods to aid in orthopedic corrective measures. For example, a “spica” is an appliance such as a rigid overlayment like a hardened bandage that is first applied in successive V-shaped crossings and used to immobilize a limb, especially at a joint. Spica devices typically comprise a splint or cast to provide stasis of a joint, i.e., immobilization for an extended period of time of convalescence.

A drawback to state of the art garments is their bespoke nature for a particular ailment or body part or even for a particular patient. Many of these garments are not modifiable such that patients are required to purchase different devices for each different ailment.

A need exists in the art for an orthotic and a method for providing orthosis for a myriad of ailments. The orthotic should be modular in design such that aspects of the orthotic may be used separately, combined, or serially. The orthotic should be adjustable and reapplied during the course of use for a single patient, even when the patient suffers from different somewhat unrelated ailments. Conversely, the orthotic should be adapted for use by several different patients having different ailments.

### SUMMARY OF INVENTION

An object of the invention is to provide a device and a method for facilitating dynamic rehabilitation protocols for muscular and skeletal (musculoskeletal) disorders that overcome many of the drawbacks of the prior art.

Another object of the invention is to provide a system and method for ameliorating musculoskeletal disorders. A feature of the invention is the use of a foundation garment in combination with one or a plurality of pressure bands reversibly attached to the foundation garment. (The garment may be shorts, long pants, a short sleeve shirt, a long sleeve shirt, overalls, or combinations thereof.) An advantage of the invention is that the same foundation garment and band(s) may be used to assist in the recovery of a myriad of different disorders during physical therapy wherein the affected area of the body is subjected to controlled movements and stretches. Another advantage is that the invention may be modified during the course of recovery for a particular patient.

Still another object of the invention is to provide a system and method for providing precise yet modifiable pressure and tactile cueing to a patient suffering from spine-, hip-, leg-, and abdomen-disorders. (Hip flexors, i.e., iliacus/psoas major/psoas minor, originate in the abdomen.) A feature of the invention is a foundation garment with tactile, visual, and auditory indicia for the reversible or variable application of pressure inducing bands. (For example, portions of the foundation garment may provide sensory cues such as surface aberrations, or sounds, e.g. tones, indicating specific muscles being targeted when palpated or overlaid by therapy bands.) An advantage of the invention is that a patient herself may apply pressure inducing bands, without the need for more experienced medical personnel. Another advantage is that the visual and/or auditory indicia may indicate the extent of therapy or the time for changing to a stronger or weaker band. The indicia may also provide an uplink (e.g., via RFID) to medical personnel for remote monitoring of treatment therapy.

Yet another object of the present invention is to provide a flexible system and method for treating skeletal ailments. A feature of the invention is the provision of a foundation garment which may or may not be worn with pressure inducing bands. An advantage of the invention is that it provides a gradient of pressure applications ranging from light to medium to high.

Briefly, the invention provides a garment for providing dynamic orthosis, the garment comprising: a foundation garment defining a crotch, a waistband, front leg regions and back leg regions; hook and pile fasteners overlying regions of the shorts; and one or a plurality of elastic bands removably received by the fasteners. For example, the substrate may have a first end removably received by first region of a garment worn by the patient and a second end removably received by a second region of the garment.

Also provided is a method for providing orthosis, the method comprising applying and/or varying pressure to affected areas of a patient overlaid with indicia. The pressure is a combination of compression applied by a foundation garment and torque applied by an elastic elongated substrate reversibly attached to the garment.

### BRIEF DESCRIPTION OF DRAWING

The invention together with the above and other objects and advantages will be best understood from the following detailed description of the preferred embodiment of the invention shown in the accompanying drawings, wherein:

FIG. 1A is a schematic elevational view of a foundation garment having horizontally arranged band attachment points, in accordance with features of the present invention;

FIG. 1B is a schematic elevational view of a foundation garment having vertically disposed band attachment points, in accordance with features of the present invention;

FIG. 2A is posterior static view of a patient wearing the foundation garment, in accordance with features of the present invention;

FIG. 2B is a posterior active view of the patient shown in FIG. 2A, in a lunging position, showing the supporting effects of stabilizer bands, in accordance with features of the present invention;

FIG. 3A shows an anterior static view of a wearer of the foundation garment, in accordance with features of the present invention;

FIG. 3B shows a right side view of a wearer of the foundation garment shown in FIG. 3A, but in an active lunge (loaded step-up) position of the left leg, in accordance with features of the present invention;

FIG. 3C shows the right side view of FIG. 3B but with the wearer in an active lunge position of the right leg (active step-up knee drive), in accordance with features of the present invention;

FIG. 4 shows a left side view of a wearer running in the foundation garment, in accordance with features of the present invention;

FIG. 5A shows the bones and musculature of the pelvis and thigh;

FIG. 5B is an anterior view of a wearer of the foundation garment showing the dynamic actions of the bone and musculature depicted in FIG. 5A, in accordance with features of the present invention;

FIG. 6 shows an anterior view of a wearer of the foundation garment in weight lifting pose (i.e., a 45 degree walking lunge), in accordance with features of the present invention;

FIG. 7A shows V shape stitching to the front of a foundation garment, in accordance with features of the present invention;

FIG. 7B shows V-shape stitching to the rear of a foundation garment, in accordance with features of the present invention;

FIG. 8A is an isometric view of a suitable band, in accordance with features of the present invention;

FIG. 8B is an isometric view of another suitable band, in accordance with features of the present invention;

FIG. 8C is an isometric view of a double spica wrap band, in accordance with features of the present invention;

FIG. 9A is a front (anterior) leg view of a hamstring therapy band positioned on a patient, in accordance with features of the present invention;

FIG. 9B is a back (posterior) view of a hamstring therapy band positioned on the back of the same leg depicted in FIG. 9A, in accordance with features of the present invention;

FIG. 10A is a view of a double spica band applied to a patient, in accordance with features of the present invention; and

FIG. 10B is a view of a double spica band and a single spica band applied to a patient, in accordance with features of the present invention.

### DETAILED DESCRIPTION OF THE INVENTION

The foregoing summary, as well as the following detailed description of certain embodiments of the present invention, will be better understood when read in conjunction with the appended drawings.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

The following detailed description should be read with reference to the drawings in which similar elements in different drawings are numbered the same. The drawings, which are not necessarily to scale, depict illustrative embodiments and are not intended to limit the scope of the invention.

As used herein, an element or step recited in the singular and preceded with the word “a” or “an” should be understood as not excluding plural said elements or steps, unless such exclusion is explicitly stated. As used in this specification and the appended claims, the term “or” is generally employed in its sense including “and/or” unless the content clearly dictates otherwise.

Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

This invention is an assistive device which a patient can use after sustaining a musculoskeletal injury in a lower extremity. It offers a combination of compression (via a foundation garment such as shorts, trousers, shirts, overalls) and torque and angle (via the application of straps or bands) to simultaneously stabilize, compress, and assist in the function of the affected muscles. (The bands may be applied in successive V-shaped crossings and used to immobilize a limb, especially at a joint.) This simultaneous application optimizes recovery, pain relief, and swelling reduction. It also serves as a device for reinforcing a wearer's lower extremity musculature during physical activity.

An embodiment of the invention is the utilization of a single band in combination with a foundation band-anchoring garment. Another embodiment is the utilization of two bands.

The invented garment is adapted to envelop at least a portion of the wearer's lumbo-pelvic region, hips, and portion of the lower extremities. Various types/levels of bands, independent of the compression short, are adjustable and allow wearer to apply bands to their desired level of support and comfort. Direction and tension of band application are dependent on the injury the wearer has sustained or the muscle group they are looking to support.

Varying levels of bands (heavy, medium, light) are serially applied and are interchangeable and generally correspond to the severity of injury and how far along the patient is in their recovery process. As she continues to recover, the wearer moves from a more supportive band (heavy) to a less supportive (light) band and eventually discontinues use of the bands altogether. Notwithstanding the patient may be left with a functional compression short.

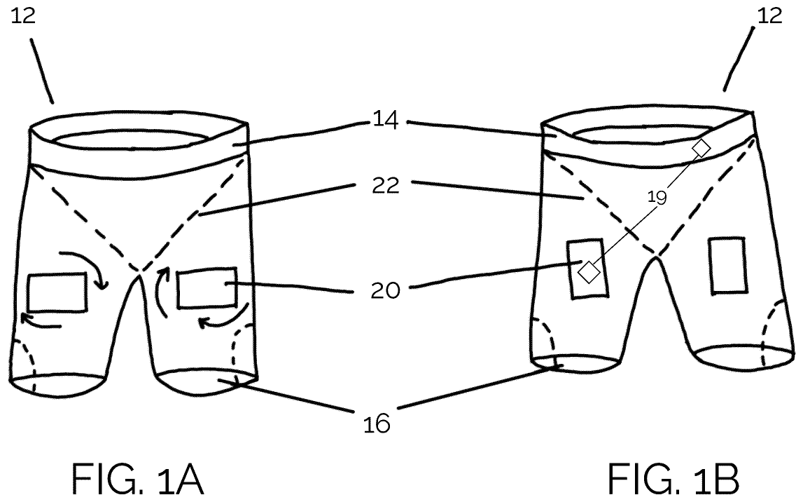

FIGS. 1A and B depict the shorts foundation (designated as numeral 12 of invented system. The foundation shorts 12 comprise a waist band 14, leg openings 16, and means 17 for reversibly attaching reversibly deformable straps or bands 18 to the foundation shorts. One such means are hook and pile (e.g., Velcro®) regions 20 such as patches positioned on the foundation shorts 12. These regions may be of various shapes such as squares, rectangles, triangles, circles, other polygrams (so named by the number of sides they have), and combinations thereof. Apices of the shapes (e.g., the corners of a rectangle or points of a triangle), or in the case of circles numbers of a clock face, may be attachment indicators of bands to aid the wearer as to where the bands should be anchored. In the case of rectangle shaped patches, the rectangles may be oriented horizontally as shown in FIG. 1A or vertically as shown in FIG. 1B. Furthermore, the patches may be oriented anywhere along a 90 degree swath from horizontal to vertical, as indicated with the curved arrows in FIG. 1A.

Generally there are at least two band attachment means to correspond to the two ends of the band. One attachment means 17 may be in the position of the above-described hook and pile region, and the other means may be along the waist band 14 of the foundation garment. As depicted in FIG. 1B, a first region of the garment (for example at the front of the leg) has visual indicia 19 (in this case a diamond) that match with second indicia at a second region (for example the waist band 14) of the garment. The indicia may be matching in shape, color and or size. Numbers may also be part of the indicia. Regions of bands intended to mate with these indicia would have the same indicia. It should be noted that inasmuch as the ends of the band may be applied to opposite sides of the foundation garment, the waist band indicia 19 may be positioned on the backside region of the waist-band.

In an embodiment of the invention the attachment means are stitched, glued, integrally molded or otherwise permanently affixed to the underlying garment. In another embodiment, the attachment points are reversibly applied for example via hook-and-pile interactions.

When oriented vertically and positioned on the front of the foundation shorts 12, the rectangular patches may be parallel with each other and flank the crotch 22 of the shorts.

Other means for reversibly attaching deformable straps include buttons and their complementary button holes, zippers, snaps and combinations thereof.

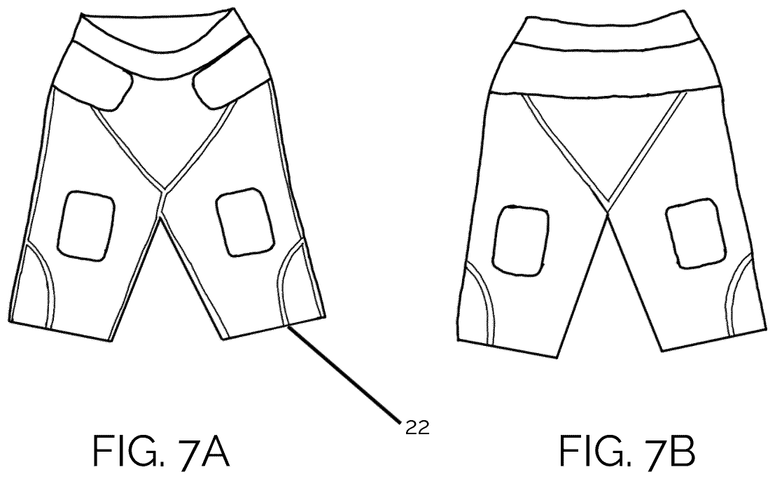

The shorts further comprise stitching 22 that confer a compressibility to them. A myriad of stitching patterns or designs are utilized to confer specific compressibility features of the foundation shorts 12. For example, FIGS. 1A-B depict stitching in cross patterns. These cross patterns provide a visual guide for the wearer as to band placement. In one embodiment of the invention, the stitching provides visual guidance for where direction of the band should be applied after being secured to the thigh. The V-shape stitching depicted in FIG. 7 starts at the crotch of the shorts. Each leg of the V extends upward and to the side so as to terminate at the outer waist of the shorts. The stitching serves as an anchor to the compressive force that is directed towards and away from the crotch. FIG. 7A shows the front view and FIG. 7B shows the posterior view of the garment.

In another embodiment of the invention, the stitching may provide a tactile indicator as to band placement; this to aid any visually impaired patients. In this instance, the stitching has a different texture or “feel” than the underlying foundation garment.

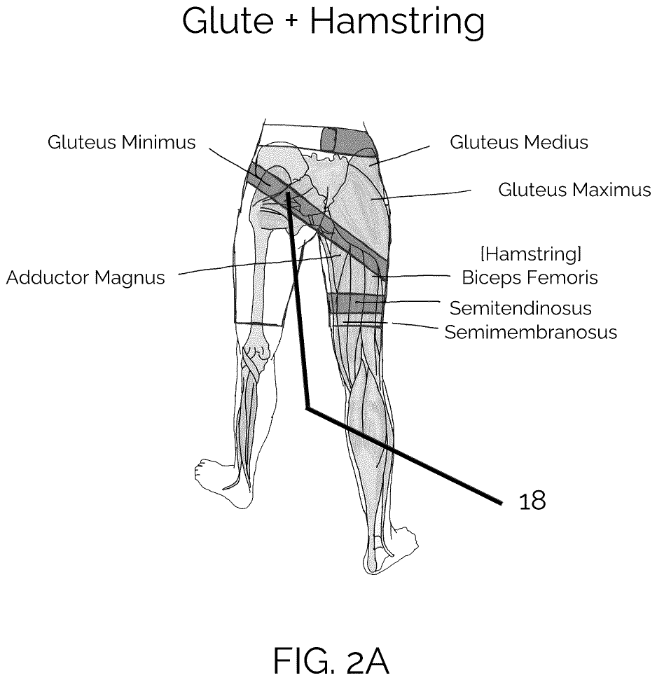

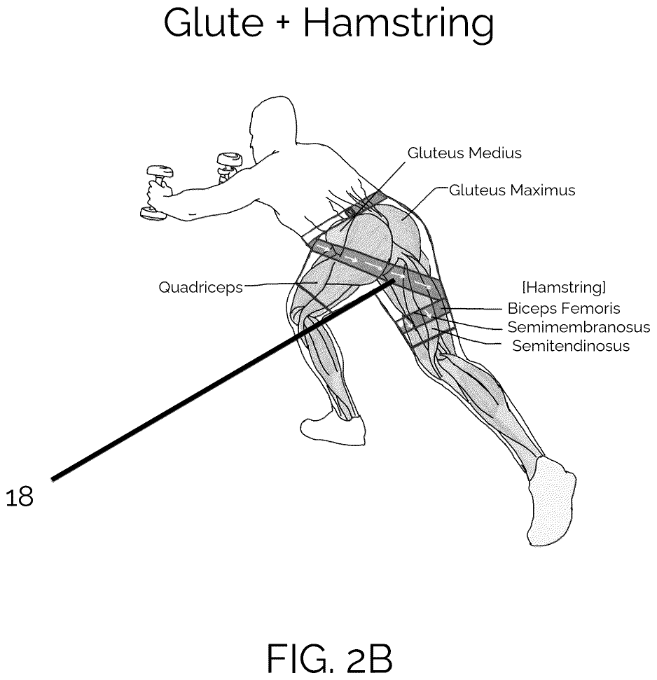

FIGS. 2A and 2B show a static and active application of the invention related to gluteal muscles (e.g., maximus, medius, minimus) and hamstring musculature (biceps femoris, semitendinosus, semimembranosus), namely isolation, support and strengthening, respectively. (It should be noted that in each of FIGS. 2A and 2B, a single band is depicted.) The diagonal portion 24 of the single band 18 in FIG. 2A is shown as relatively inactive compared to FIG. 2B (e.g., slightly stretched and imposing limited force on the patient) such that no arrows of force are depicted). However, FIG. 2B shows the single band imposing posterior downward and diagonal force and support to both the gluteus maximus and hamstring muscles (gluteal and hamstring muscles/musculature into hip extension); that force depicted by the arrows superimposed on the bands 18. The band as positioned assists the wearer into an extension position that the wearer may not otherwise have the strength or flexibility to attempt.

This tension on the band is created during hip flexion, internal rotation, and adduction of the aforementioned muscle groups. The band supports hip extension, external rotation, and abduction. Specifically, a diagonally disposed portion 24 of the band comprises a first end anchored to the wearer's mid-right thigh region and a second end anchored to the lateral aspect of the wearer's left hip. This order more accurately represents how the band is attached, creates the directionality of force, and imparts a pulling force toward that mid-right thigh region. In an embodiment of the method, initial band attachment starts at the thigh, then the band encircles the thigh 1 to 2 times. After which, the band is pulled diagonally across the body. The reversibly attachable band points allow for added emphasis of certain muscle groups. For example, if the band is attached relative to its horizontal counterpart at less than a 45 degree angle, then more adduction of the left leg muscle groups are emphasized. It should be noted that if the opposite leg is being treated, then the band would be similarly fastened to that leg and hip. Two bands may be used to simultaneously treat both sides of the patient's body.

FIGS. 2A and 2B further show a generally horizontal disposed portion 26 of the band positioned above the wearer's right knee imposing a generally vertical force downwardly toward the wearer's knee and away from the diagonally disposed band portion 24. The arrows on the horizontal portions of the bands don't create a downward force. Rather, the horizontal portion creates a level of compression and also assists in creation of the posterior force that aids in hip extension.

In summary, the band in FIG. 2B imposes a posterior directed force that aids the affected leg into extension. It resists hip flexion and adduction and aids in hip extension and abduction (thereby supporting the hamstrings and glutes).

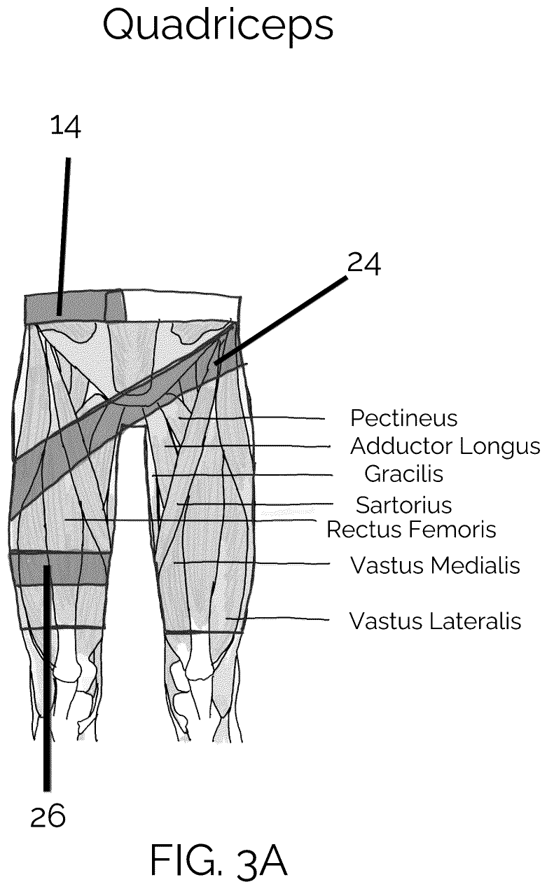

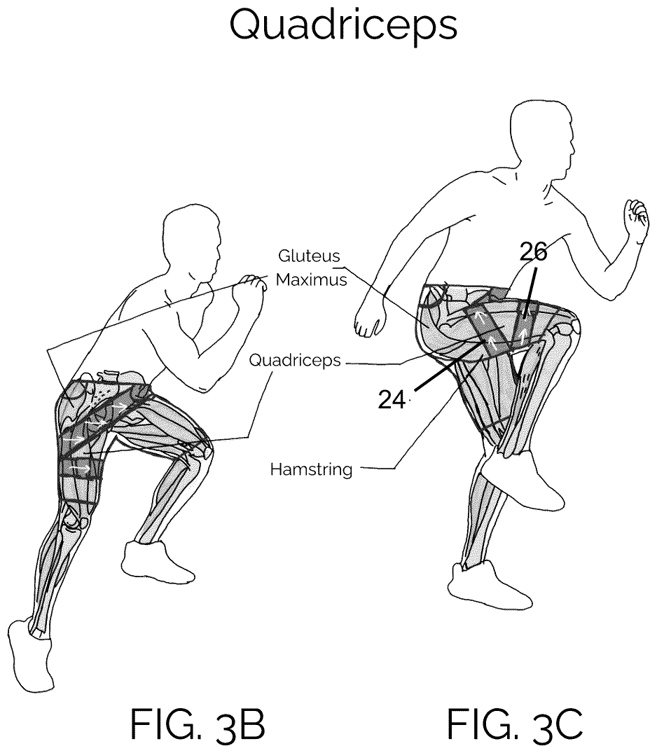

Remaining FIGS. 3-6 provide examples where the band imposes tension during hip extension, external rotation, and abduction. As such, the band supports hip flexion, internal rotation, and adduction.

For example, FIGS. 3A-3C depict the invention in quadricep, gluteus, and hamstring therapy applications. The single band positioned in FIGS. 3A-C include a horizontally disposed portion 26 of the band positioned above the right knee of the wearer and a diagonally disposed portion 24 of the band with a first end proximal to the horizontal portion and physically contacting the horizontal portion in the hamstring region. A second end of the band proximal to the diagonal portion 24 terminates at the waist band 14 region of the foundation shorts 12. FIG. 3A shows that termination point at approximately the umbilicus region of a patient, but termination can be anywhere along the periphery encircling the patient defining the waist band.

As above, the first figure (FIG. 3A) of the series shows the wearer in a static, non-active position. FIG. 3B shows the wearer with his left leg flexed upwardly. His right leg is supported by the band in hip extension.

FIG. 3C shows the wearer with his right leg flexed upwardly in hip flexion.

The superimposed arrows of the bands in FIG. 3B indicate an anterior force imposed on the hip girdle of the wearer and across the front of the foundation shorts 12. When the affected leg (right leg) is in hip extension, the quadriceps and iliopsoas (hip flexors) are eccentrically contracted. The band is providing resistance to hip extension and the tension created will assist those muscle groups into forward hip flexion (concentric contraction). Adductors (groin musculature) have a secondary role in hip flexion.

The superimposed arrows of the bands in FIG. 3C indicate anterior directed force from the back to the front of the wearer's flexed right leg. This has the effect of lengthening the hamstring muscle. As such, the band is assisting in the concentric contraction (shortening) of the quadriceps and iliopsoas.

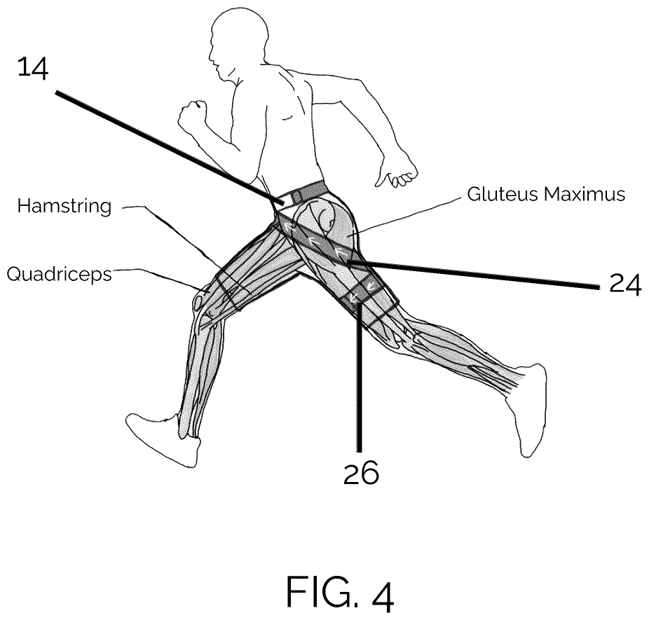

FIG. 4 shows the left side of a wearer of the same foundation garment depicted in FIG. 3A, but with the wearer in full stride with his right leg forward and his left leg trailing. The arrows superimposed on the diagonally disposed portion 24 of the band show an anteromedial directed force, thereby aiding the affected (left) leg into hip flexion and adduction. Arrows depicted on the horizontally disposed portion 26 of the band provide a generally upward directed, anterior force, thereby stabilizing the hamstring. The effect of these two band portions is to maintain the positions of the glutes and hamstrings to avoid over extension of these two muscle groups during running, while having relatively less effect on posterior musculature. This is similar to the band placement shown in FIG. 3B resists hip extension and abduction and aids in hip flexion and adduction. The band applied in this manner assists adductors, iliopsoas, and the quadriceps.

Inasmuch as the bands are reversibly attached to the foundation garment, the band may be arranged to allow for greater stabilization, for example with the lower end of the diagonal portion 24 of the band positioned further behind the wearer. Anterior and posterior attachment sites on the thighs offer the wearer a means to customize their desired level of compression and tension in the band.

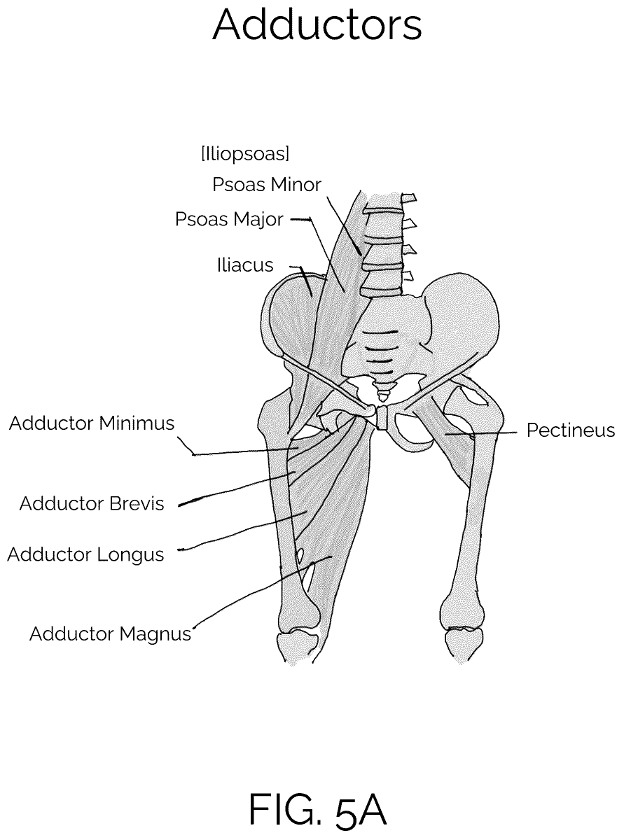

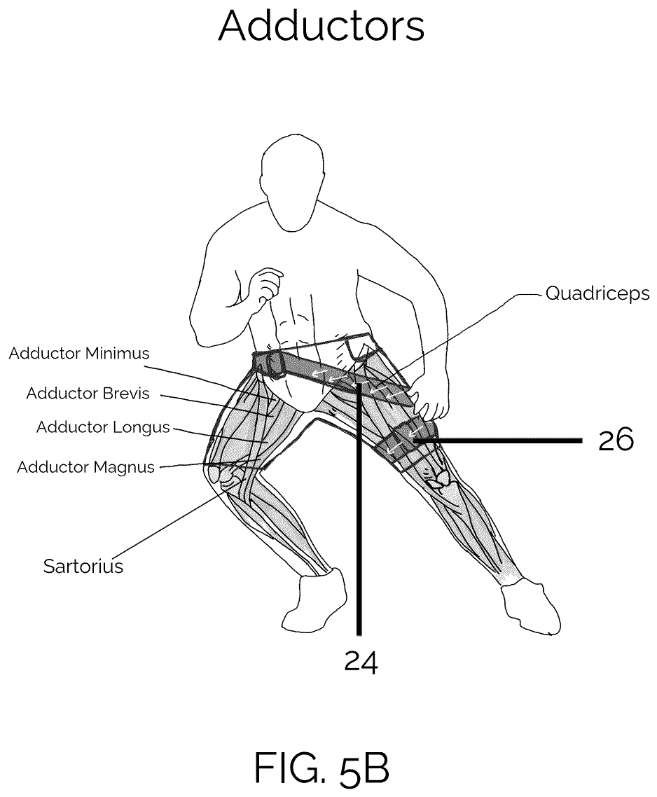

FIG. 5A shows the skeletal underlayment and muscular overlayment of adductors in the groin region.

FIG. 5B shows the effect of the foundation garment on those musculoskeletal structures. The superimposed arrows of both the diagonal portion 24 and horizontally arranged portion 26 of the band show a midline bias of the bands to aid in adduction of the groin muscles, thereby minimizing abduction of those muscles. Depending on how the bands reversibly attached to the foundation garment will determine the effect of those forces. For example, if the horizontally arranged portion 26 of the band is fastened lower on the leg portion of the foundation garment, greater force from greater stretching would result, such that abduction would be further restricted. Such are the benefits of the bands reversible attachment features to a plurality of different fastener points. As such, more than one fastener point may exist on the foundation garment at each end of the band to affect this pressure variability.

Furthermore, this pressure variability may be electronically communicated to a remotely situated caregiver. For example, mobile phone monitoring of such physiological stress points utilize sensors embedded in a smart phone, wrist band, and or the foundation garment to detect pressures experienced by the body, such as skin tightness (electrodermal sensors), movement patterns (e.g. accelerometers), blood flow, blood pressure, and combinations of these data points. As such, real-time monitoring of these pressure levels are possible through an app or other software.

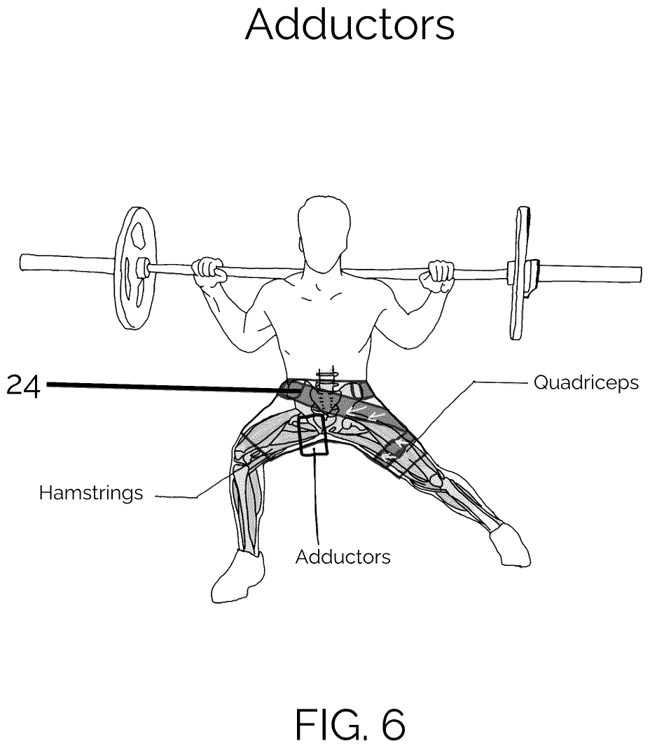

FIG. 6 is an anterior view of a weight lifter wearing the foundation garment-band construct. The diagonal portion 24 and horizontally disposed portion 26 of the band show a general midline bias. This feature stabilizes the hamstrings, quadriceps and the adductors of the groin (namely the adductor minimus, brevis, longus, magnus, and gracilis muscles).

### Band Detail

Generally, the band is of sufficient length to wrap around a patient's body part while also being anchored to the band fastening means discussed supra. In an embodiment of the invention, the band may be 3 to 4 inches wide and 4 to 5 feet in length.

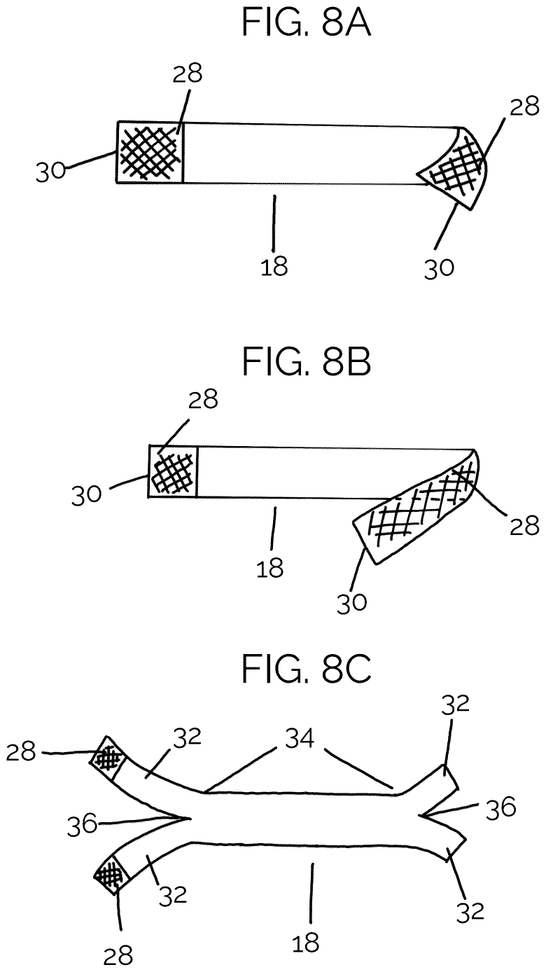

FIG. 8A is an isometric view of a suitable band 18, in accordance with features of the present invention. FIG. 8A shows an elastic band, which may have dimensions of between 3 and 4 inches wide, 40-48 inches long and 1/32-⅛′ thick, preferably 1/16″ thick. The band also features complementary hook-and-pile fasteners 28 at its ends 30. The fasteners are situated on opposite ends of the band. These fasteners are similar in shape (e.g., rectangular or square), similar in width and length and are oriented the same way on the same side of the band. In an embodiment of the invention, both ends of the bands terminate in hooks for mating with complementary hoops or loops at respective anchor points on the foundation garment. Conversely, both ends of the bands terminate in hoops or loops for mating with complementary hooks at respective anchor points on the foundation garment.

FIG. 8B is an isometric view of a double spica wrap band, wherein the band may be wrapped around the patient's leg twice. As with the configuration of FIG. 8A, this configuration features two hook and pile fastener regions 28 at the ends 30 of the band and on opposite sides thereof. As in FIG. 8A, one side has the hook portions on both ends. Then on one end, on the opposite side of the band is a longitudinally longer section covered in hoops/loops. In summary of this point, each end of the band has both hook and loop portions. The longer loop portion will be exposed when applied but such that this longer fastener region may be approximately three to four times longer than that at the opposite end of the band. This, to allow the band to be wrapped around the patient's leg more than once to confer twice the fastening strength, or additional fastener surface for another band.

For example, when the band is applied, the longer portion of hoop/loop will be exposed. This allows a second band to be applied to the other leg such that the larger area is now a new attachment site option up on the hip for the second band to attach. The second band that can be attached may be a “regular” band like FIG. 8A.

In an embodiment of the invention, the hook portion of the fastener is positioned on the side of the band that is intended on facing forward from the front of the patient while the hook portion of the fastener at the other end of the band is positioned on the side of the band that would be facing away from the back side of a patient. In this embodiment, both ends have hooks on the same side like FIG. 8A. For this band, on the opposite side of the hooks, one end features a complementary loop portion. The hook side of band may still be used for attachment to the foundation garment. When the band is applied, the loop side should be exposed on the outside. This allows additional attachment site space to accommodate a second band applied to the opposite limb. The initial band may be covering the hip attachment site so this FIG. 8B band allows a second band to still have somewhere to attach to the hip if needed. When using this band, the end that doesn't have the loop portion should be applied first around the thigh.

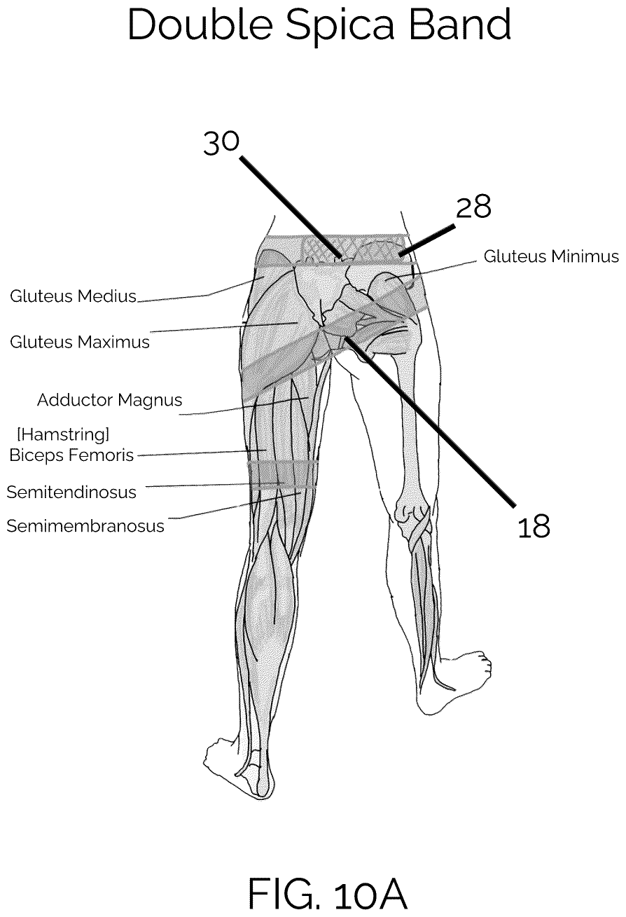

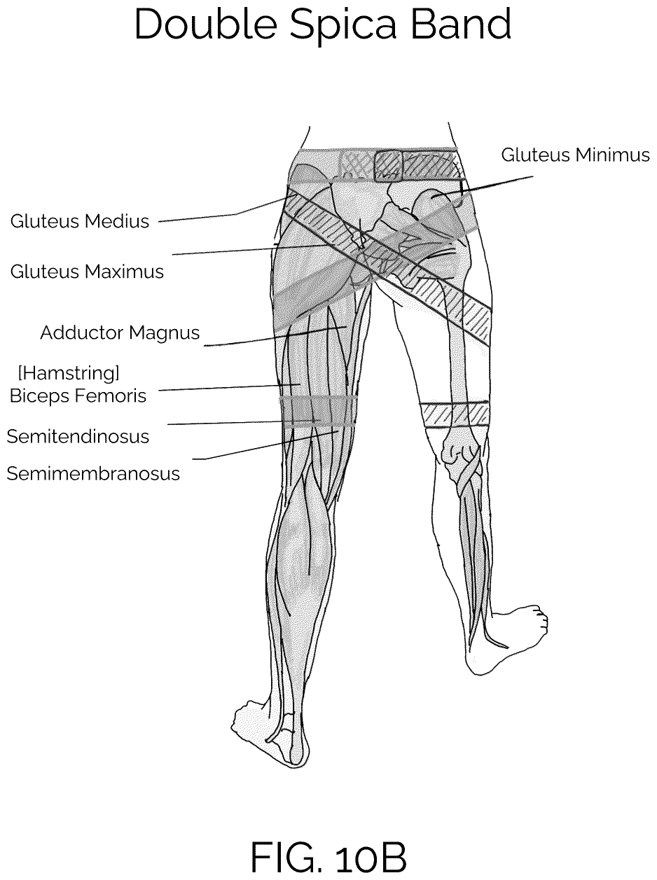

FIGS. 10A and 10B show the double spica band at work. FIG. 10A shows the double spica band laid first on the patient. Here, the double sided hook and pile (e.g. Velcro®) band is attached to the foundation garment (e.g., shorts, as shown) and awaits the attachment of the regular spica band depicted in FIG. 8A.

FIG. 10B shows the regular spica band (shown in a fluted pattern) laid over the double spica band where it attaches to the back side of the Velcro. This allows for the patient to be supported on both areas (e.g., the hips and the groin areas) while providing enough support for the patient to proceed in regular ranges of motion.

FIG. 8C is an isometric view of what is dubbed a hamstring band. Unlike the iterations shown in FIGS. 8A-B, FIG. 8C is not generally rectangular but rather shaped as an elongated X, whereby the X is situated horizontally with two of the four legs 32 of the X extended slightly upward and the remaining two directed slightly downward. The length of this band is between 15 and 30 inches, preferably between 20 and 25 inches. An embodiment of the invention is between 20 and 22 inches long. A center portion 34 of the band is approximately 5-10 inches in length between the two forked regions 36 of the X. A preferred center portion 34 length is between 6 and 8 inches long. A preferred embodiment has a center region that is approximately seven inches long.

The length of the forked regions 36 at each end of the band may be similar or different. FIG. 8C shows the forked regions 36 on its left longer than the forked region on its right. When the band is applied, the longer arms on the band would be superior and attach to the waist. The shorter arms would be inferior and are attached to the thigh. The shorter arms wrap from the posterior aspect of the thigh and attach to the anterior attachment site. The band is then pulled up vertically and the superior arms can be stretched and attached to the waist. Longer levers for the arms provide more strength/stability when attaching.

Opposing surfaces of the forks may be reinforced with strengthening materials such as hemp, linen, wool, cotton, carbon fiber, and combinations thereof, this to prevent the band from splitting down the middle of the fork when tension is applied to the forks. The strengthening materials may also comprise the same material used in band construction. The materials may be radio-transparent to allow imaging analyses of the patient during band application.

Hook and pile fasteners are positioned at distal portions of each of the tips of the forked regions. Hooked portions of the fasteners 28 are situated on the wearer facing outwardly from the front or anterior side of the patient while pile portions are situated relative to the wearer facing away from the back or dorsal side of the patient. This arrangement is depicted in (FIGS. 10A and 10B.) These Velcro patches may be generally square and with dimensions from 1-4 inches, preferably 2-3 inches. In an embodiment of the invention, all four arms have hooks positioned on their distal portions, and all on the same side of the band.

The bands and/or the foundation garment may indicate (tactilely, visually or auditorily, the later via a series of tones or beeps) the amount of force the bands impose when attached to various regions of the garment. For example, the foundation garment may indicate the Newton values required for band placement at a particular region of the foundation garment. (Exemplary Newton values are featured in Table 1, below.) Alternatively, Newton values may be colorized so that a colored region of the garment is overlaid with the same color code on the band. This allows untrained patients to apply and remove the appropriate bands during the course of their therapy, without the need for intervention by trained personnel. In addition, electronic uplinks from bands, once applied to the garment, to trained personnel will provide an added layer of scrutiny to band application.

TABLE 1
Polyester Band 48 inches in length

| Extent of Stretch | Force Applied in Newtons |
|---|---|
| (% beyond relaxed length) | (N) |
| 10% | 6.9 |
| 20% | 9.7 |
| 30% | 11.3 |
| 40% | 13.5 |
| 50% | 15.9 |

Polyester Band 60 inches in length″

| | |
|---|---|
| 10% | 7.4 |
| 20% | 10.3 |
| 30% | 13.2 |
| 40% | 16.1 |
| 50% | 17.1 |

Nylon Band 48 inches in length

| | |
|---|---|
| 10% | 7 |
| 20% | 14.3 |
| 30% | 18 |
| 40% | 21.6 |
| 50% | 26.5 |

Nylon Band 60 inches in length

| | |
|---|---|
| 10% | 7.8 |
| 20% | 14.1 |
| 30% | 19.2 |
| 40% | 23.6 |
| 50% | 28.6 |

Generally, the bands are made of materials with varying levels of elasticity and lengths. For example, bands having light contraction strength are comprised of polyester. Bands having medium to heavy contraction strength are comprised of nylon polyester and natural latex rubber. In an embodiment of the invention, light contraction may comprise Newton resistance values greater than 0 and less than 12. Medium contraction may comprise Newton resistance values between 12 and 18. Heavy contraction may comprise Newton resistance values greater than 18. So, in a practical application of this dynamic treatment, Newton resistance values of greater than 18 may be applied for a prescribed period of time; then lower contraction values of say 12-15 N may be applied, then finally light contraction values of greater than 0 and less than 12 may be applied. The patient would be wearing the same foundation garment, with only different bands being applied.

The bands were tested at Oct. 20, 1930/40/50 percent stretch of their total length. For example, if the band is 10 inches long unstretched, then a 10% stretch results in an 11″ long band. The bands were subjected to 25 trials. The forces to the right of the stretch percentage figures represent pull back force in Newtons and are calculated averages of the trials. Table 1 above shows the results of these tests.

The aforementioned band characteristics may be highlighted to a user via the use of indicia, including tactile, auditory, and visual. Tactile indicia may include topographic artifacts such as braille or other type of surface aberrations. Visual indicia may include printing, color coded signals, matching shapes, and a combination of these. As discussed supra related to FIG. 1B, the first end of the band may have first indicia that match with first indicia at the first region of the garment and the second end of the band may have second indicia that match with second indicia at the second region of the garment.

Auditory indicia may include a motion sensor to announce or otherwise advise a user of the type of band they are harvesting. These indicia may include single audio cues such as beeps or tones, or prerecorded messages in the selected language of the wearer.

The utilization of auditory signals may also include the transmission of acoustic signals known to emanate from the body during manipulation. These acoustic signals may be used as a basis for determining correct band placement or the immediate effect of such placement. This automated analysis may utilize a large audio data set capturing the application of manipulation types on different body locations to provide immediate acoustic feedback to the user.

For example, in orthopedics, acoustic signals have been used for diagnosis and guidance. Vibroarthgraphy (VAG) is characterized by recording acoustic emission from knee joints in order to detect malicious or subclinical joint conditions. Various degrees of chondromalacia and meniscal lesions can be detected by performing a frequency analysis on the audio signal recorded with surface microphones mounted to the patient's skin, or to the foundation garment.

### Operation Detail

### Single Band Configuration:

In operation, application of the device to the patient begins by applying one end of the band to the attachment site on the thigh. The band is wrapped 1-2 times around the thigh and pulled at an angle to wrap around the waist.

In a groin/hip flexor application, the loose end of band is pulled medially around thigh and then across the front of the waist at an angle to the contralateral hip. Anatomical landmark: AIIS (anterior inferior iliac spine).

For hamstring/glute therapy, one begins by applying one end of the band to the attachment site on the anterior thigh. The loose end of the band should sit laterally and wrapped around the thigh 1-2 times and pulled at an angle around the ipsilateral (same side) hip. Both applications can be started on the posterior anchor band attachment point (e.g., a Velcro patch situated there).

A hamstring specific treatment band is depicted in FIG. 8C. This “X-shaped band is described supra. It is applied from the posterior aspect of the thigh. The bottom straps will be pulled around posterior to anterior and secured to the anterior thigh attachment site.

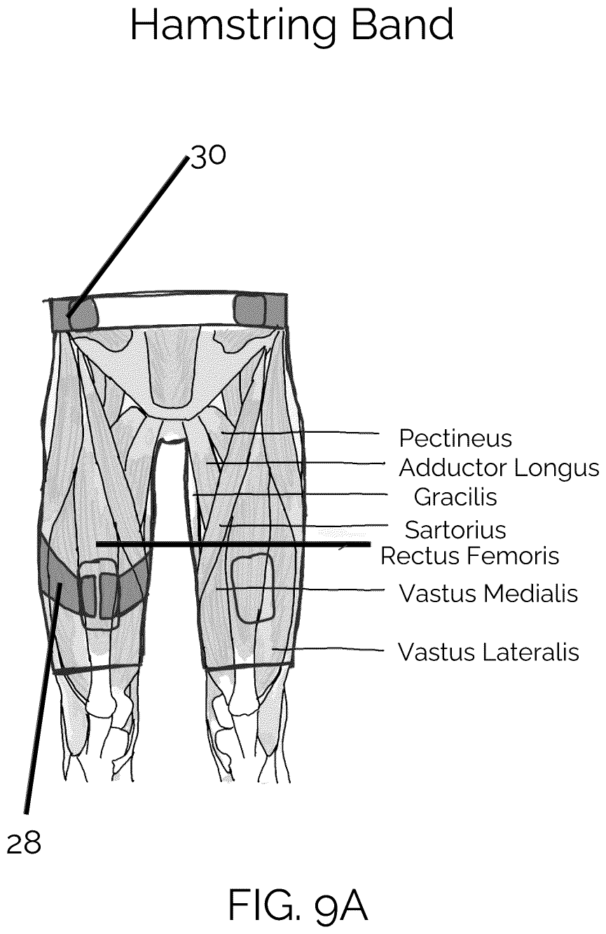

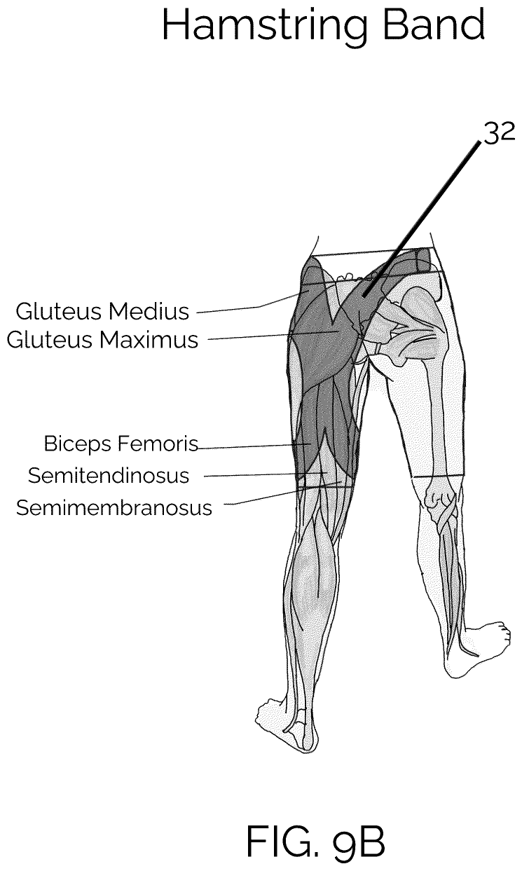

FIG. 9A is a front leg view of a hamstring treatment band positioned on a patient. FIG. 9B is a posterior view of a hamstring treatment band positioned on the back of the patient. FIG. 9A shows a vertically disposed hook and pile anchor patch irreversibly or reversibly attached to the front of the foundation garment. This patch may be moved from one leg to the other, depending on which hamstring needs therapy.

As shown in FIGS. 9A-B, the shorter forked legs are first anchored just above of one of the patient's knee Then the longer forked legs depicted in FIG. 8C are positioned superior of the shorter forked legs on the patient and these top straps are pulled upward and secured to the hip attachment site.

In summary, in a preferred use, a single band iteration of the device is applied with a first end of the band first being applied to a first anchor point at the leg or thigh. Then, the band is stretched or otherwise extended via its second end to a second anchor point. As discussed supra, the wearer starts with a heavier gauge band to a less supportive one over the course of her treatment; thus an embodiment of the dynamic nature of the invention. One end of the band will be secured to the anterior or posterior thigh attachment site (depending on injury or intended use), wrapped around the thigh once or twice, then pulled up and around the waist where the other end of the band will be secured to the hip attachment site.

### Multiple Band Configuration:

Another embodiment utilizes two bands, for example, one anchored on each leg. In this embodiment, a first band with Velcro on both sides and a loop positioned at or near one end. That end is applied first to the hip with the loop side facing outwards so as to provide an attachment point for a second belt around the hip, if needed. (The first band will likely be covering the original attachment point supplied by the foundation garment at that location, so the loop provides a second attachment site option.).

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting, but are instead exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the terms “comprising” and “wherein.” Moreover, in the following claims, the terms “first,” “second,” and “third,” are used merely as labels, and are not intended to impose numerical requirements on their objects. Further, the limitations of the following claims are not written in means-plus-function format and are not intended to be interpreted based on 35 U.S.C. § 112, sixth paragraph, unless and until such claim limitations expressly use the phrase “means for” followed by a statement of function void of further structure.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” “more than” and the like include the number recited and refer to ranges which can be subsequently broken down into subranges as discussed above. In the same manner, all ratios disclosed herein also include all sub ratios falling within the broader ratio.

One skilled in the art will also readily recognize that where members are grouped together in a common manner, such as in a Markush group, the present invention encompasses not only the entire group listed as a whole, but each member of the group individually and all possible subgroups of the main group. Accordingly, for all purposes, the present invention encompasses not only the main group, but also the main group absent one or more of the group members. The present invention also envisages the explicit exclusion of one or more of any of the group members in the claimed invention.

1. A system for providing orthosis, the system comprising:
a. a foundation garment defining a crotch, a waistband, front leg regions and back leg regions;
b. fasteners overlying the regions of the garment; and
c. a reversibly deformable elongated substrate removably received by the fasteners.

2. The system as recited in claim 1 wherein the fasteners each define a longitudinal axis and at least one of the fasteners is positioned with its longitudinal axis extending upwardly toward the head of a patient and downwardly toward the feet of the patient when the garment is worn by the patient.

3. The system as recited in claim 1 wherein a mid-point of at least two of the fasteners is in line with the crotch of the garment.

4. The system as recited in claim 1 including stitching extending from the crotch to a lateral region of the waistband, whereby the stitching imposes a compressive force and whereby the stitching provides visual guidance for where direction of the substrate should be applied.

5. The system as recited in claim 1 wherein varying levels of strengths of the removable substrate are adapted to be serially applied to provide light elasticity, medium elasticity and heavy elasticity.

6. The system as recited in claim 1 wherein the fasteners are attached to the front leg regions and the back leg regions.

7. The system as recited in claim 6 wherein longitudinal axes of the fasteners are parallel to each other.

8. The system as recited in claim 1 further comprising substrate attachment indicia on the foundation garment.

9. The system as recited in claim 8 wherein the indicia correspond to substrate characteristics selected from the group consisting of newton resistance, length, muscle group applicability, and combinations thereof.

10. The system as recited in claim 8 wherein the indicia comprise stitching on the garment as a tactile indication of where to attach the substrate.

11. The system as recited in claim 8 wherein the indicia provide a sensory cue selected from the group consisting of tactile, visual, auditory, compression, and combinations thereof as to the correct location of substrate placement on the garment.

12. The system as recited in claim 8 wherein the indicia provide a signal as to whether a correct substrate is being applied.

13. A system for providing orthosis, the system comprising:
a. a foundation garment defining a crotch, a waistband, front leg regions and back leg regions;
b. fasteners overlying regions of the garment; and
c. a reversibly deformable elongated substrate received by the fasteners; the system further comprising substrate attachment indicia wherein the indicia provide a signal as to whether the correct substrate is being applied, wherein the substrate band is in electronic communication with the indicia.

14-20. (canceled)

21. The system as recited in claim 5 wherein the removable substrates comprising light elasticity have Newton resistance values greater than 0 but less than 12, the removable substrates comprising medium elasticity have Newton resistance values between 12 and 18, and the removable substrates with heavy elasticity have Newton resistance values greater than 18.

22. The system as recited in claim 13 wherein the indicia provide a sensory cue selected from the group consisting of tactile, visual, auditory, compression, and combinations thereof as to the correct location of substrate placement on the garment.

23. The system as recited in claim 13 wherein the substrate provides light elasticity, medium elasticity and heavy elasticity.

24. The system as recited in claim 13 wherein the fasteners are attached to the front leg regions and the back leg regions.

25. The system as recited in claim 13 wherein longitudinal axes of the fasteners are parallel to each other.
